(19) United States
(12) Patent Application Publication
(10) Pub. No.: US 2013/0079692 A1
(43) Pub. Date: Mar. 28, 2013

(54) INFLATABLE COMPRESSSION SLEEVE

(71) Applicant: Mego Afek AC Ltd., Doar Afek (IL)

(72) Inventor: Asher BEN-NUN, Carmiel (IL)

(73) Assignee: MEGO AFEK AC LTD., Doar Afek (IL)

(21) Appl. No.: 13/680,714

(22) Filed: Nov. 19, 2012

Related U.S. Application Data
(63) Continuation-in-part of application No. 12/555,356, filed on Sep. 8, 2009, now Pat. No. 8,313,450.
Application No. 12/555,356 is a continuation-in-part of application No. 10/895,292, filed on Jul. 21, 2004, now abandoned.

(51) Int. Cl. A61F 5/01

(52) U.S. Cl. CPC A61F 5/012
USPC 602/23; 156/252; 601/151

(57) ABSTRACT

A disposable sleeve for compression therapy including a first wall to be located adjacent a patient's body to be treated and a second wall to be spaced from the patient body by the first wall, each of the walls comprising an internal layer made of an airtight material and an external layer disposed adjacent to the internal layer and made of a porous material, the internal layers of the two walls facing each other, all of the layers bonded to each other along bonding seams and free of such bonding at locations spaced from the bonding seams; and airtight cells defined between the walls, bound by the bonding seams and configured for being inflated, and at least one non-inflatable area spaced from adjacent cells by at least one bonding seam and having one or more through holes formed in the internal layer of each of the walls.

### CROSS-REFERENCE TO RELATED APPLICATIONS

This application is a continuation-in-part of U.S. patent application Ser. No. 12/555,356 filed on 8 Sep. 2009, which is a continuation-in-part of U.S. patent application Ser. No. 10/895,292, filed on 21 Jul. 2004, the contents of each of the foregoing applications are incorporated herein, in their entirety, by this reference.

### TECHNICAL FIELD

Embodiments of the invention relate to inflatable sleeves for use in pneumatic compression therapy and to methods of producing such sleeves.

### BACKGROUND

Deep vein thrombosis, also known as DVT, is a serious and potentially life-threatening disorder. The physiological cause of this disorder is lack of adequate blood circulation in the lower extremities. The lack of movement of venous blood can cause clots to form, which may cause blockages in the local blood vessels, or in more serious situations, may lodge in the lungs or heart and cause critical blockages which can be life-threatening.

A large number of medical research studies have shown that deep vein thrombosis can be prevented by enhancing or accelerating the return of venous blood from the lower extremities. A common and accepted method for accelerating venous blood return from the lower limbs is pneumatic compression applied to the sole of the foot and/or the calf muscle of the leg. This form of treatment is commonly referred to as “compression therapy,” and is performed using a compression device, which feeds compressed air to a garment or “sleeve” containing one or more cells which inflate and deflate, alternately applying and releasing pressure to the patient's lower extremities.

In hospitals, there are many devices of this kind, and there are compression therapy usage protocols for patients who are hospitalized for operative procedures or have other risk factors for developing deep vein thrombosis. The compression therapy devices may be used 24 hours a day for the entire hospitalization period. Clinical studies have shown that the effectiveness of such devices is primarily determined by patient and staff compliance, which in turn is affected by ease of use and patient comfort. The usage of such devices is also determined by economic factors such as cost of the device and garments as opposed to pharmaceutical interventions such as heparin.

U.S. Pat. No. 4,013,069 describes compression sleeves made of interior impervious sheets and one or more sheets of soft flexible material for covering the outside of the impervious sheets adjacent the patient's leg. The outer sheets may be made of any suitable material, such as TYVEK™, and they provide an aesthetically pleasing and comfortable outer surface for the sleeve. The outer sheets may be attached to the internal sheets by suitable means, such as stitches along the side and end edges. The sleeves may have a plurality of hook and loop strips to releasably secure the sleeves about the patient's legs.

U.S. Pat. No. 4,066,084 describes a cuff comprising a piece of stable fabric or plastic material of soft but not elastic quality, in the shape of a trapezium, the two non-parallel sides having the same length. The two non-parallel sides are provided with a divisible zip fastener, by means of which the cuff can be shaped to a slightly tapered cylinder fitting a patient's limb. On one side of the form-stable material, a number of elongated inflatable sections are provided arranged parallel to the parallel sides of the trapezium. These sections are manufactured of an elastic, strong plastic material, rubber or other air impervious material. The sections may also consist of balloons inserted in pockets in the cuff.

U.S. Pat. No. 4,338,923 describes a sleeve wrappable about the body part to be treated, made in the form of a substantially flat inflatable band divided into a plurality of internal inflatable cells extending annularly around the sleeve, in partially overlapping relationship. The band is made of three strips of resilient sheet material bonded to each other along spaced bond lines to define the partially overlapping inflatable cells.

The above-described sleeves for compression therapy are of durable construction and constitute a constant part of the massaging device that is used multiple times with different patients, mostly as physical therapy for chronic venous and lymphatic disorders.

When these devices are used as prophylaxis for deep vein thrombosis, either in the operating theater or during the recovery period, the specific needs of the hospital market are for disposable, one-time or one-patient use sleeves. Such made from PVC fabric are manufactured by the Kendall Co. (Tyco) as well as by other major manufacturers. However, the cost of these sleeves is still high, and hospitals have had to reprocess and reuse these so-called “disposable” sleeves in an attempt to cut expenses. In addition, PVC is now considered an environmentally “unfriendly” material, and its use has been curtailed in many countries because of concerns of carcinogenicity. The PVC outer layer also prevents normal evaporation of perspiration, causing discomfort to the patient.

### SUMMARY

In accordance with one aspect of the presently disclosed subject matter there is provided a disposable sleeve for compression therapy, the sleeve having a first wall to be located adjacent a patient's body to be treated and a second wall to be spaced from the patient body by the first wall, each of said first and said second walls comprising an internal layer made of an airtight material and an external layer disposed adjacent to the internal layer and made of a porous material, the internal layers of the two walls facing each other, the walls with all their layers being bonded to each other along bonding seams and being free of such bonding at locations spaced from the bonding seams; the sleeve comprising a plurality of airtight cells defined between the first and second walls, bound by the bonding seams and configured for being inflated, and at least one non-inflatable area spaced from adjacent cells by at least one bonding seam and having one or more through holes formed in the internal layer of each of the walls.

In accordance with another aspect of the presently disclosed subject matter there is provided a method of producing a disposable sleeve, the sleeve having at least one inflatable air cell defined between a first and a second walls, each of said walls including an external layer of porous material and an internal layer made of an airtight material, the method comprising:

a) providing a first and a second layer made of an airtight material;

b) providing a first and a second layer made of porous material;

c) forming in said first and second layers made of the airtight material through holes at first areas spaced from second, airtight areas which are free of said holes and which correspond to said cells;

d) arranging said layers in a flat stack so that the two layers made of the airtight material have their first and second areas aligned and are sandwiched between the two layers of porous material;

e) bonding said flat stack of the layers along seams to form said inflatable air cells and non-inflatable areas including said holes.

### BRIEF DESCRIPTION OF THE DRAWINGS

In order to understand the subject matter and to see how it may be carried out in practice, embodiments will now be described, by way of non-limiting examples only, with reference to the accompanying drawings, in which:

FIG. 1 is a view of a compression therapy sleeve according to an embodiment of the present subject matter, fixed in operative condition on a patient's lower limb.

FIG. 2 is a plan view of the sleeve of FIG. 1 in flat condition.

FIG. 3A is a side view of the sleeve of FIG. 1 in folded condition.

FIG. 3B is a cross-sectional view of the lower section of the sleeve of FIG. 3A.

FIG. 4 is a schematic cross-section through an air cell of the sleeve of FIG. 1.

FIG. 5 is an enlarged cross-section of a welding zone in the sleeve of FIG. 4.

FIG. 6 is a schematic cross-section through an air cell of a sleeve according to another embodiment of the present subject matter.

FIG. 7A is a view of a sleeve according to another embodiment of the present subject matter.

FIG. 7B is a schematic cross-sectional view through a wall of the sleeve of FIG. 7A at an area thereof which is free of an air cell and which comprises aligned holes in its inner sheets.

FIG. 8 is a schematic cross-section through an air cell of a sleeve according to another embodiment of the present subject matter.

### DETAILED DESCRIPTION OF THE EMBODIMENTS

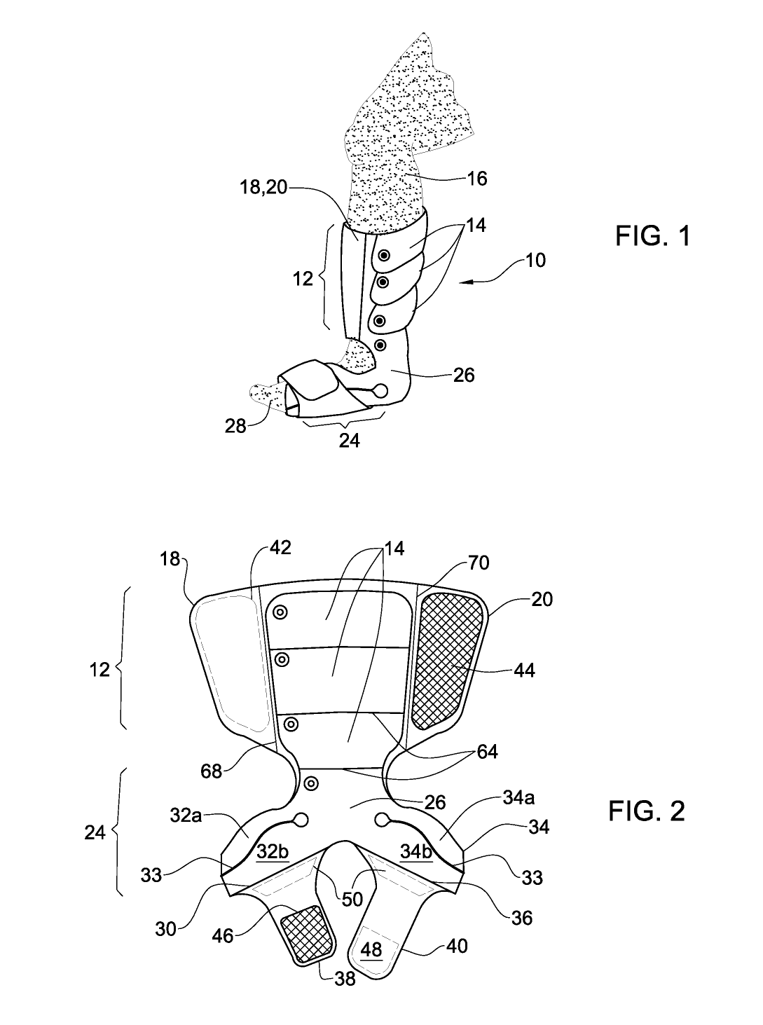

With reference to FIGS. 1, 2 and 3, a disposable compression sleeve 10 in accordance with one embodiment of the present subject matter, designed for prevention of

DVT, comprises an upper section 12 with upper air cells 14 for compressing the patient's calf 16, with upper fastening flaps 18 and 20 formed at left and right sides of the air cells 14; a lower section 24 with lower air cell 26 for compressing the sole 28 of the patient's foot, with lower fastening flaps 38 and 40; and air inlets (nipples) 41 in the air cells 14 and 26 mounted for connecting the air cells, by means of hoses, to an inflating device such as a compressor with distributor valve (not shown).

In the upper section 12, the size of the air cells 14 is not designed to cover only the calf muscle 16 rather than the whole circumference of the limb. The rest of the circumference is bridged by the fastening flaps 18 and 20, as explained below. The flaps 18 and 20 may be formed from the material of the air cells 14 or may be attached thereto along seams 68 and 70.

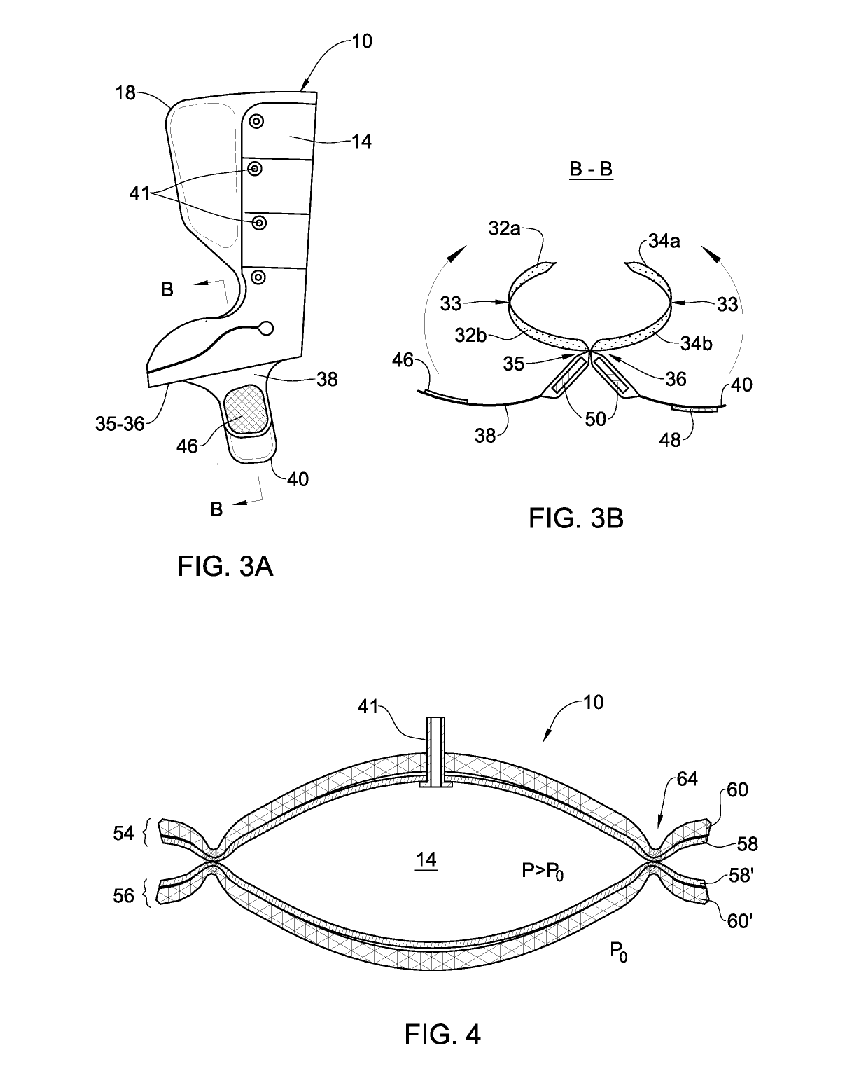

In the lower section 24, the lower air cell 26 has a left part 32 and right part 34 divided by seams 33 into upper lobes 32a and 34a, and lower lobes 32b and 34b, respectively. The air cell 26 has a left lower edge 35 and a right lower edge 36, shown unassembled in FIG. 2. In assembled state, the edges 35 and 36 are bonded together, whereby the lower lobes 32b and 34b form an inflatable sole, as shown in cross-section in FIG. 3B. The lower fastening flaps 38 and 40 are attached to the same edges 35 and 36. The fastening flaps may be formed integral with the lower section, from the sheet material of the sleeve. In such case, the edges 35 and 36 will be just seams between the air cell 26 and the flaps 38 and 40.

The upper section 12 and the lower section 24 of the sleeve 10 may be manufactured as one-piece garment but may be also separate and be used as two separate units.

In operative position, the sleeve 10 is placed against the foot of the patient with the upper section 12 behind the calf and the lower section 24 under the heel and sole of the foot. The air cells 14 are wrapped about the calf and fastened by means of the flaps 18 and 20. The lower section 24 is wrapped about the foot and fastened over the instep by means of flaps 38 and 40. Lobes 32b and 34b remain adjacent the sole of the foot while lobes 32a and 34a are adjacent the sides and the instep of the foot.

The fastening flaps 18, 20, 38 and 40 provide for closure and fastening of the sleeve around a wide range of limb girths without disrupting or affecting the air cells, thus eliminating the need for a variety of sizes for this sleeve. The fastening of the flaps may be realized by various means, for example hook and loop Velcro patches 42, 44, 46 and 48. Also, the fastening of the flaps may be effected by means of a self-adhesive layer on the flaps with the adhesive side protected by removable tape. Alternatively, a separate two-sided self-adhesive patch can be used, which can be placed on the sleeve by the patient or treatment personnel for closure according to the patient's limb exact size.

In another embodiment of the sleeve, the lower section 24 of the sleeve 10 may also contain rigid plates 50 (FIG. 2) built into the flaps 38 and 40, adjacent the seams 35 and 36, so as to support the lobes 32b and 34b that are in contact with the sole 28 of the foot. The rigid plates at the sole of the foot apply a force-resistant surface to the air cell, improving the efficiency of application of pressure to the sole of the foot. The rigid plates 50 may be insertable in pockets formed in the lower fastening flaps 38 and 40 adjacent the right and left lower lobes 32b and 34b of the lower air cell 26. In all the above cases, the rigid plate(s) 50 may be made of a stiff plastic, such as a board made of PVC, or other materials. When it is desired to prevent multiple use of the sleeve 10 and washing thereof required for such use, the plates 50 may be made of a material such as, for example, cardboard, which looses its rigidity when wetted.

With reference to the cross-section shown in FIG. 4, an air cell 14 or 26 in the sleeve 10 is formed with an upper wall 54 and a lower wall 56, where the lower wall 56 is adjacent the patient's limb when the sleeve is in use. The walls 54 and 56 comprise each a respective inner sheet 58, 58′ and a respective outer sheet 60, 60′ bonded together along lines 64, 68, 70, 33, 35, 36, etc. defining the contours of the air cells (only line 64 is seen in cross-section in FIG. 4). The inner sheets 58 and 58′ are made of polyethylene, for example metallocene PE of Dow Chemicals, which is relatively cheap. The material is well weldable and airtight though not particularly strong. However, the inventors have tested and proved that, for example, a 100-150 μm sheet of this material has sufficient tensile strength and durability for a guarantied limited number of inflation-deflation cycles. This number is typically about 30,000 for a few days of pre-surgery or post-surgery treatment of one patient. The number may be considerably less, about 250 for one or two procedures of compression therapy, which allows the usage of even thinner sheets of PE. The requirements to the cell walls strength may be further reduced if the cells do not embrace the whole circumference of the limb but about two-thirds or less. That is why, this material is very suitable for making disposable sleeves used for prevention of DVT in the limbs. The outer sheets 60 and 60′ are made of porous material such as textile fabric. Preferably, non-woven textile is used, for example polypropylene or polyester fabric.

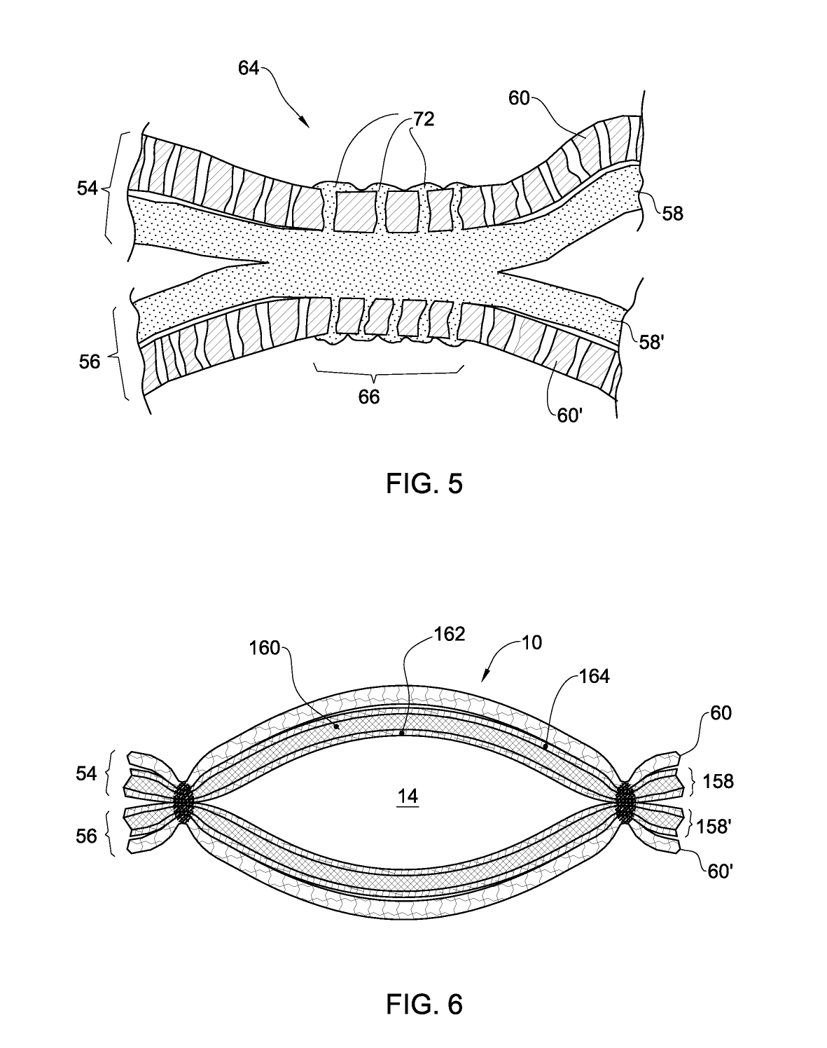

The bonding of the constituent sheets is done in a special way shown in FIG. 5. The two PE sheets 58, 58′ are welded to each other, in a welding zone 66, for example by RF heating. At the same time, molten portions 72 of the PE in the welding zone 66 penetrate the pores of the porous material 60 and solidify there, locking the outer sheets 60, 60′ to the PE sheets 58, 58′ and to each other. Notably, the porous material need not be weldable to the PE layer. The inventors have discovered that such bonding may be sufficiently reliable and provides the required durability for the same number of cyclic inflations-deflations as above.

A method for production of the disposable compression therapy sleeve above includes the following steps:

a) providing an inner sheet 58 made of PE and an outer sheet 60 of porous material for the upper wall 54, cutting them to suitable form, aligning them and inserting air nipples 41 in openings of the sheets 58, 60;

b) bonding the air nipples 41 to the inner PE sheet and to the porous sheet 60;

c) providing an inner sheet 58′ and an outer sheet 60′ for the lower wall 56 and cutting them to a suitable form;

d) aligning the four sheets of material in a flat stack (Velcro pads, male and female, may be provided, with backside laminated with PE layer or with a porous layer, and aligned in the same flat stack. Also plates of stiff plastic 50 may be provided and inserted between the sheets);

e) bonding the stacked sheets across the stack along a pattern of seams 33, 35, 36, 64, 68, 70, etc. defining air cells 14 and 24;

f) folding the stack and bonding the left and right parts 32 and 34 of the lower air cell 26 together along their lower edges 35 and 36 to form a scoop-like accommodation for the heel of the foot, as shown in FIGS. 3A and 3B.

The fastening flaps 18, 20, 38 and 40 may be formed as extensions of the sleeve walls 54, 56 beyond the air cells so that the flaps will be obtained simultaneously with the air cells at step (e).

It is possible that all seams in the compression sleeve are obtained in one bonding stroke including welding, melting and setting. The bonding stroke may be combined with a cutting operation, for example, to obtain the outer contour of the sleeve.

As shown in FIG. 6, the compression therapy sleeve 10 may be made of reinforced inner sheets 158, 158′ of more complex structure. The sheet 158 or 158′ may comprise for example a reinforcing non-woven or nylon layer 160 sandwiched between two polyethylene layers 162 and 164, formed as an integral sheet, for example by lamination. Such materials are manufactured for use in the food packaging industry and are relatively cheap. The overall thickness of the inner sheets in this case may be even less than of a purely PE inner sheet. It will be appreciated that the same method of bonding as above can be applied.

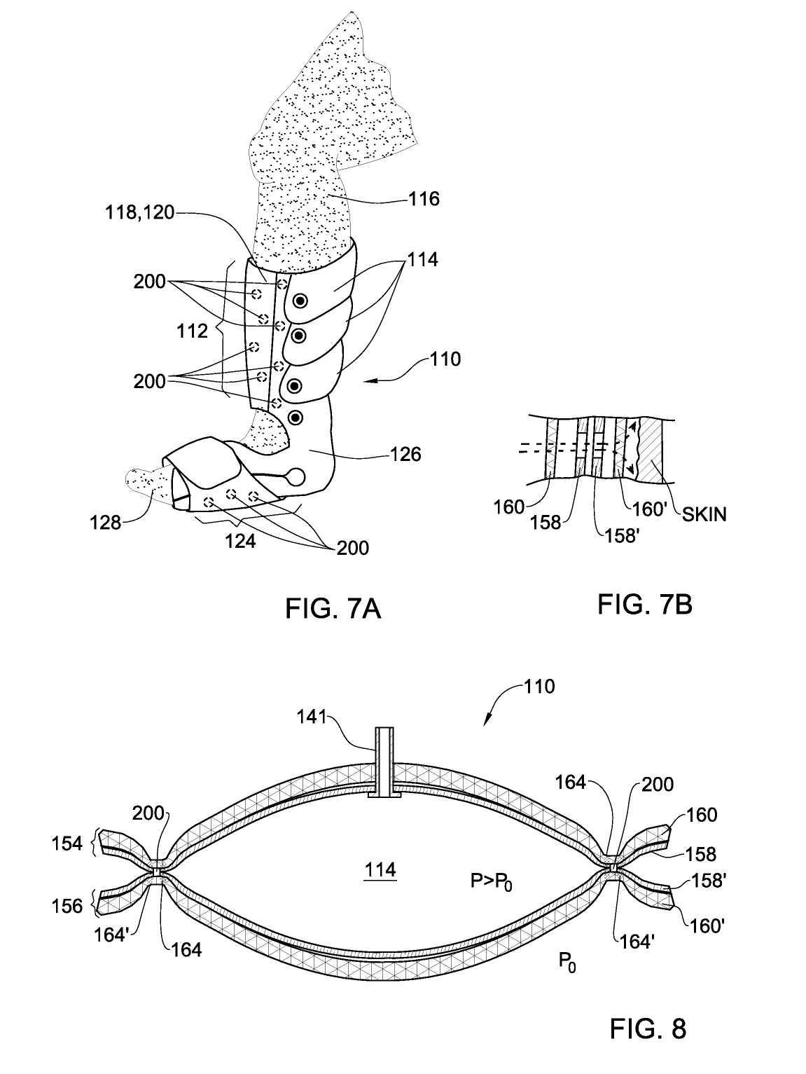

With reference to FIGS. 7A, 7B and 8, there is shown a disposable compression sleeve 100 in accordance with another embodiment of the presently disclosed subject matter. The sleeve 100 comprises an upper section 112 with upper air cells 114 for compressing the patient's calf 116, with upper fastening flaps 118 and 120 formed at left and right sides of the air cells 114; a lower section 124 with lower air cell 126 for compressing the sole 28 of the patient's foot, with lower fastening flaps 138 and 140; and air inlets (nipples) 141 in the air cells 114 and 126 mounted for connecting the air cells, by means of hoses, to an inflating device such as a compressor with distributor valve (not shown).

With reference to FIGS. 7A and 8, an air cell 114 or 126 in the sleeve 100 is formed with an upper wall 154 and a lower wall 156, where the lower wall 156 is configured to contact the patient's limb when the sleeve is in use. The walls 154 and 156 comprise each a respective inner sheet 158, 158′ and a respective outer sheet 160, 160′ bonded together along several bonding seams (of which only seams 164 and 164′ are seen in cross-section in FIG. 8). The inner sheets 158 and 158′ are made of an airtight material, such as polyethylene, and can be further reinforced with PE loop-formed fibers. The outer sheets 160 and 160′ are made of porous material such as textile fabric. Preferably, non-woven textile is used, for example polypropylene or polyester fabric.

The sleeve 100 further comprises a plurality of through holes 200 formed in the inner sheets 158 and 158′, at non-inflatable areas A, which, in the context of the present invention can be any portion of the sleeve other than an air cell 114, 126, i.e. free of such air cell. The non-inflatable areas can comprise, among others, areas adjacent to the bonding seams, areas between bonding seams (FIG. 8), fastening means, such as the flaps 18 and 20, etc. The outer sheets 160 and 160′ are free of holes corresponding to holes 200. In this connection, it should be explained that in FIG. 7B the holes 200 are shown in dotted lines to illustrate their location under the outer sheets 160 and 160′.

The holes 200 are significantly larger than the pores in the outer sheets 160 and 160′, and have distances therebetween greater than the diameter of the holes. In particular, the distances between the holes can be at least not less than twice the diameter of the holes.

As shown in FIG. 8, at least some of the air cells 114 and 126 can have two bonding seams 164 and 164′ therebetween, which are spaced one from the other along the entire thickness thereof by a separating area, at which the holes 200 can be formed in the inner sheets 158 and 158′.

In operation, the inflation of the air cells causes the non-inflatable areas of the sleeve to become spaced from the patient's body, which results in the air from the exterior of the sleeve being drawn into such spacing via the pores in the outer sheets and the holes in the inner sheets, as shown by arrows in FIG. 7B. In other words, when an air cell is inflated, a vacuum is created at areas between the patent's body and the sleeve at areas other than the air cell, allowing an ambient air to enter to these areas through the pores in the outersheets and the holes in the inner sheets. By virtue of this, there is provided a to ventilation and moisture evaporation from the patient's skin adjacent to the sleeve during the inflation and deflation cycles thereof.

In order to achieve a better spacing, the holes can be disposed in a closed proximity of the bonding seams and the air cells.

A sleeve with holes 200 as described above can be produced in the same manner as the sleeve without the holes, with the difference being in that the process can include the following steps:

a) forming in the inner sheets made of the airtight material through holes at first areas spaced from second, airtight areas which are free of such holes and which correspond to the air cells;

b) arranging the sheets in a flat stack so that the two inner sheets have their holes aligned and are sandwiched between the outer sheets; and

c) bonding the flat stack of the sheets along seams to form the inflatable air cells and non-inflatable areas including the holes.

It should be noted that surprisingly the use of the pores of the outer sheets have appeared to provide higher effectiveness for the air circulation than the use of holes in non-porous outer sheets.

Although a description of specific embodiment has been presented, it is contemplated that various changes could be made without deviating from the scope of the present subject matter. For example, the present subject matter could be modified and used for production of other compression devices for treating DVT or lymphedema.

1. A disposable sleeve for compression therapy, comprising:
a first wall to be located adjacent a patient's body to be treated;
a second wall to be spaced from the patient body by the first wall, each of the first and the second walls including an internal layer made of an airtight material and an external layer disposed adjacent to the internal layer and made of a porous material, the internal layers of the first and second walls facing each other, the first and second walls having all of the layers thereof bonded to each other along bonding seams and being free of such bonding at locations spaced from the bonding seams; and
a plurality of airtight cells defined between the first and second walls, bound by the bonding seams and configured for being inflated, and at least one non-inflatable area spaced from adjacent cells of the plurality of airtight cells by at least one of the bonding seams and having one or more through holes formed in the internal layer of each of the walls.

2. The disposable sleeve according to claim 1, wherein the at least one non-inflatable area occupies a majority of space in the disposable sleeve that is free of the cells.

3. The disposable sleeve according to claim 1, wherein at least some of the one or more through holes are disposed adjacent to the bonding seams.

4. The disposable sleeve according to claim 1, wherein at least some of the adjacent cells are spaced one from the other by two or more of the bonding seams so that some of the one or more through holes are disposed between the two or more of the bonding seams.

5. The disposable sleeve according to claim 1, further comprising fastening means for fixing the disposable sleeve on the patient's body.

6. The disposable sleeve according to claim 5, wherein the fastening means includes at least one flap made of the same material as the disposable sleeve and including at least one hole in the internal airtight layer thereof.

7. The disposable sleeve according to claim 1, wherein the airtight material includes polyethylene.

8. The disposable sleeve according to claim 1, wherein the internal layer is reinforced with polyethylene loop-formed fibers.

9. A method of producing a disposable sleeve, the disposable sleeve having inflatable air cells defined between first and second walls, each of the first and second walls including an external layer of porous material and an internal layer made of an airtight material, the method comprising:
providing first and second layers made of an airtight material;
providing first and second layers made of porous material;
forming in the first and second layers made of the airtight material through holes at first areas spaced from second, airtight areas that are free of the through holes and which correspond to the inflatable air cells;
arranging the layers in a flat stack so that the two layers made of the airtight material have their first and second areas aligned and are sandwiched between the two layers of porous material; and
bonding the flat stack of the layers along seams to form the inflatable air cells and non-inflatable areas including the through holes.

10. The method according to claim 1, wherein the non-inflatable areas occupy a majority of space in the disposable sleeve that is free of the inflatable air cells.

11. The method according to claim 1, wherein at least some of the through holes are disposed adjacent to the bonding seams.

12. The method according to claim 1, wherein at least some adjacent inflatable air cells of the inflatable air cells are spaced one from the other by two or more bonding seams so that some of the through holes are disposed between the two or more bonding seams.

13. The method according to claim 1, further comprising fastening means for fixing the sleeve on the patient's body.

14. The method according to claim 13, wherein the fastening means includes at least one flap made of the same material as the sleeve and including at least one hole in the internal airtight layer thereof.

15. The method according to claim 1, wherein the airtight material includes polyethylene.

16. The method according to claim 1, wherein the internal layer is reinforced with polyethylene loop-formed fibers.
